(19) United States
(12) Patent Application Publication
(10) Pub. No.: US 2014/0336464 A1
(43) Pub. Date: Nov. 13, 2014

(54) EXPANSION DEVICES AND METHODS OF USE THEREOF

(71) Applicant: Boston Scientific Scimed, Inc., Maple Grove, MN (US)

(72) Inventor: Laura E. FIRSTENBERG, Worcester, MA (US)

(73) Assignee: Boston Scientific Scimed, Inc., Maple Grove, MN (US)

(21) Appl. No.: 14/269,423

(22) Filed: May 5, 2014

Related U.S. Application Data
(60) Provisional application No. 61/820,937, filed on May 8, 2013.

(51) Int. Cl. A61B 18/14

(52) U.S. Cl. CPC A61B 18/14; A61B 2018/00982
USPC 600/160; 606/45

(57) ABSTRACT

Devices, systems, and methods for facilitating access to and visualization of the pancreaticobiliary system are disclosed. In particular, the present disclosure relates to devices used to expand and/or maintain the papilla opening to facilitate access therethrough, e.g., to visualize, examine, diagnose, and/or treat the bile duct and pancreatic ducts. The devices may include an elongate body, an expansion element having a compressed configuration and an expanded configuration, and an outer sheath.

### CROSS-REFERENCE TO RELATED APPLICATIONS

This application claims the benefit of priority from U.S. Provisional Application No. 61/820,937, filed on May 8, 2013, the entirety of which is incorporated by reference herein.

### TECHNICAL FIELD

Various embodiments of the present disclosure relate generally to medical devices and related methods of use thereof. More specifically, the present disclosure relates to devices and methods for accessing the pancreaticobiliary system, e.g., to examine, diagnose, and/or treat a condition of the pancreatic duct or the bile duct.

### BACKGROUND

Access to the pancreaticobiliary system is required to diagnose and/or treat a variety of conditions, including tumors, gallstones, infection, sclerosis, and pseudocysts. One method of gaining access is via endoscopic retrograde cholangiopancreatography (ERCP), in which a side-viewing endoscope is passed down the esophagus, through the stomach, and into the duodenum where the duodenal papilla leading into the pancreatic and bile ducts may be visualized. In ERCP, tools such as sphincterotomes are passed through the working channel of the scope to gain access to the papilla, e.g., to investigate potential obstruction or inflammation of the pancreatic or bile ducts. Fluoroscopic contrast may be injected into either duct and X-ray images taken to determine the presence and location of strictures or stones.

Cannulation of either the bile duct or the pancreatic duct is a significant challenge in ERCP procedures. Factors that may complicate insertion into the papilla include sphincter orientation, floppy intraductal segments, biliary/pancreatic take-off levels, and the presence of stones or strictures. Difficult cannulations carry a high risk of perforation or other damage to tissue. For example, one technique physicians use to cannulate the papilla is to identify a bile trail, e.g., by pushing against the ampulla to encourage bile from the duct. Prolonged probing, however, may lead to inflammation of the papilla and adverse effects for the patient.

Complications also may arise when the duct accessed first is not the duct desired for the procedure. When biliary access is desired, for example, a physician first may gain access to the pancreatic duct, e.g., via a guidewire. The physician then would have to remove the wire and attempt cannulation again. The pancreatic duct may be entered unintentionally several more times before access to the bile duct is finally achieved. These multiple pancreatic injections can irritate the tissue of the pancreatic duct and cause post-ERCP complications such as pancreatitis.

Thus, there remains a need for alternative methods of accessing the pancreaticobiliary system in order to improve efficacy of medical treatment and increase patient safety.

### SUMMARY OF THE DISCLOSURE

The present disclosure includes devices and methods of use thereof for accessing the pancreaticobiliary system and enhancing visualization therein, such as during an ERCP procedure.

Embodiments of the present disclosure include a method of accessing the pancreaticobiliary system, the method comprising: introducing a distal end of an instrument into at least a portion of a duodenal papilla, the instrument including: an elongate body having a proximal end, a distal end, and at least one lumen extending therebetween; and an expansion element circumferentially disposed around the elongate body, the expansion element having a compressed configuration and an expanded configuration; and deploying the expansion element into the expanded configuration, wherein in the expanded configuration, the expansion element expands against a wall of the papilla. Embodiments of the present disclosure may include one or more of the following features: the method may comprise introducing a guidewire into at least a portion of the papilla before introducing the instrument into the papilla; the expansion element may be self-expanding; the instrument may include a sheath circumferentially disposed around the expansion element, wherein deploying the expansion element includes moving the sheath in a proximal direction to uncover the expansion element; the expansion element may include a metal mesh; the elongate body may be a sphincterotome having an electrosurgical cutting wire; the method may comprise advancing the elongate body in a distal direction; the method may comprise using an optical device to image a feature of the pancreaticobiliary system; the method may comprise performing a medical procedure within at least one of the pancreatic duct and the bile duct with the treatment instrument; or the method may comprise removing the expansion element from the papilla.

Embodiments of the present disclosure also include a medical device comprising: an elongate body having a proximal end, a distal end, and at least one lumen extending therebetween, the elongate body configured for insertion into a duodenal papilla; and an expansion element circumferentially disposed around the elongate body, the expansion element having a compressed configuration and an expanded configuration suitable for insertion into and enlarging the duodenal papilla, wherein a distal end of the expansion element is located proximal to the distal end of the elongate body. Embodiments of the present disclosure may include one or more of the following features: the medical device may comprise a sheath circumferentially disposed around the expansion element and the elongate body, wherein the sheath is slidable with respect to the expansion element; the expansion element may include a metal, a polymer, or a combination thereof; the expansion element may include an expandable metal mesh; the expansion element may include a coating; the expansion element may be fixed to the elongate body; the expansion element may be movable with respect to the distal end of the elongate body; the elongate body may include a retractable hook for grasping a portion of the expansion element; the expansion element may be configured for atraumatic contact with a tissue surface in the expanded configuration; or the elongate body may be a sphincterotome having an electrosurgical cutting wire.

### BRIEF DESCRIPTION OF THE FIGURES

The accompanying drawings, which are incorporated in and constitute a part of this specification, illustrate various exemplary embodiments and together with the description, serve to explain the principles of the disclosed embodiments.

FIG. 1 shows anatomical features of the pancreaticobiliary system.

FIGS. 2A-2B show an exemplary device, in accordance with the present disclosure.

FIGS. 3A-3D illustrate a method of accessing the pancreaticobiliary system, in accordance with the present disclosure.

FIGS. 4A-4B show an exemplary device, in accordance with the present disclosure.

### DETAILED DESCRIPTION

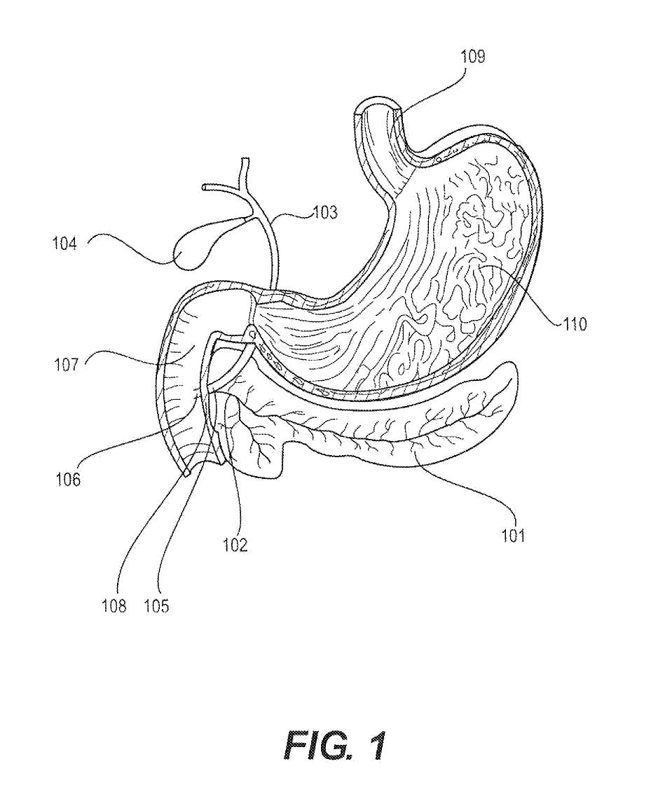

The pancreaticobiliary system, illustrated in FIG. 1, includes the pancreas (101), the pancreatic duct (102), the common bile or biliary duct (103), and the gallbladder (104). The pancreatic and biliary ducts join at the hepatopancreatic ampulla (105) (also known as the ampulla of Vader), which lies just behind the major duodenal papilla (106). The papilla (106) is a small opening that leads into the duodenum (107) to allow for the release of pancreatic juice and bile into the duodenum to aid in digestion. Smooth muscle of the hepatopancreatic sphincter (108) (also known as the sphincter of Oddi) regulates flow of pancreatic juice and bile into the duodenum. The minor duodenal papilla (not shown) is a separate small opening in the duodenum, upstream of the major papilla (106), that leads into the accessory pancreatic duct. The minor papilla is usually nonfunctional (i.e., does not release pancreatic juice into the duodenum) and may be absent, for example in patients lacking an accessory pancreatic duct. While the present disclosure generally relates to the major duodenal papilla (referred to herein simply as “papilla”), it is understood that the present disclosure also may be useful in accessing the minor duodenal papilla.

Referring again to FIG. 1, in an ERCP procedure, an endoscope may be passed down the esophagus (109), through the stomach (110), and into the duodenum (107) to gain access to the pancreatic duct (102) and/or bile duct (103) via the papilla (106). The passageway leading from the papilla (106) towards the pancreatic duct (102) and bile duct (103) tends to be tortuous and difficult to navigate, however, e.g., via a guidewire, catheter, or other medical device. In some patients, the papilla may also be obscured from view by a diverticulum. A physician may make several unsuccessful attempts at cannulation, increasing the risk of injury to the patient, before access is achieved. In addition, a physician may not enter the desired duct initially, e.g., due to an inability to visualize and/or distinguish each of the pancreatic and bile ducts.

According to embodiments of the present disclosure, a device including an expansion element may be introduced into the papilla to facilitate access to the pancreaticobiliary system. For example, the expansion element may support, anchor, and/or enlarge the papilla to enhance visualization and facilitate the passage of one or more instruments therein.

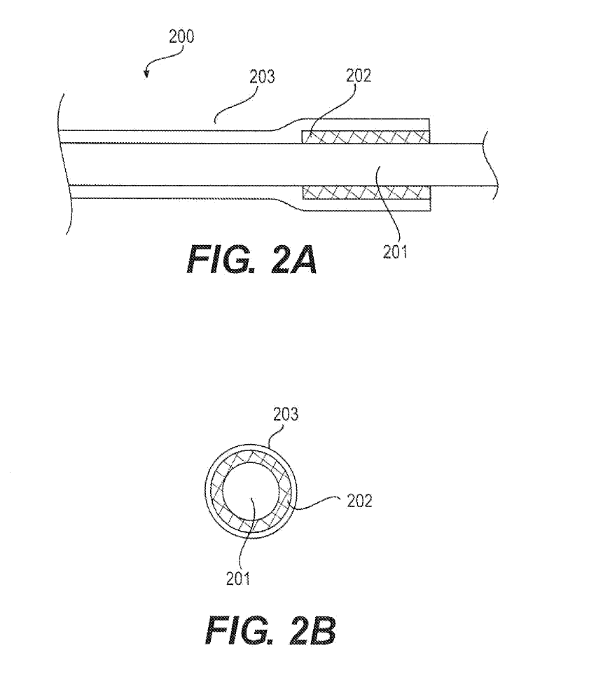

According to an embodiment illustrated in FIG. 2A, the device (200) may comprise an elongate body (201) having a proximal end, a distal end, and one or more lumens extending therebetween. The distal end of the elongate body (201) may include an expansion element (202) and a sheath (203) circumferentially disposed around the expansion element (202). FIG. 2B is a cross-section of FIG. 2A, showing a concentric configuration of the elongate body (201), expansion element (202), and sheath (203) of FIG. 2A. The cross-section need not be circular, however, but may have any suitable regular or irregular cross-sectional shape including, e.g., oval, rectangular, square, etc. The sheath (203) may surround or enclose all or only a portion of the expansion element (202).

The expansion element (202) may be disposed between the elongate body (201) and the sheath (203), for example constrained along an outer surface of the elongate body (201) by the outer sheath (203). In some embodiments, the expansion element (202) is located along a portion of the distal end of the elongate body (201) proximate the distal most end as shown in FIG. 2A. In other embodiments, the expansion element (202) may be located at the distal most end, for example, or proximate the location shown in FIG. 2A. The expansion element (202) may be fixedly attached or moveable with respect to the elongate body (201). For example, the expansion element (202) may be slidable along the outer surface of the elongate body (201). In some embodiments, the expansion element (202) may be slidable with respect to the elongate body (201) only when unconstrained by the sheath (203). For example, placement of the sheath (203) around the expansion element (202) may establish a friction fit such that the frictional force may not be overcome due to the constraining effect of the sheath (203). In other embodiments, the expansion element (202) may be slidable with respect to the elongate body (201) even when surrounded by the sheath (203).

In some embodiments of the present disclosure, the sheath (203) may extend along substantially the entire length of the elongate body (201), i.e., from the proximal end to the distal end, wherein the sheath (203) surrounds at least a portion of the expansion element (202) at the distal end of the elongate body (201). In other embodiments, the sheath (203) may extend along only a portion of the elongate body (201).

The expansion element (202) may be compressible, e.g., having a compressed configuration and an expanded configuration. FIG. 2A, for example, shows the expansion element (202) in a compressed configuration constrained between the elongate body (201) and the sheath (203). The sheath (203) may be moveable with respect to the elongate body (201), e.g., slidable in a proximal and/or distal direction. Sliding the sheath (203) in a proximal direction may uncover the expansion element (202) and allow it to expand from a compressed configuration to an expanded configuration. Once the sheath (203) is moved in a proximal direction to uncover the expansion element (202), the expansion element (202) may expand radially outward.

In some embodiments, the expansion element (202) is self-expanding, requiring no external stimulus to affect expansion once unconstrained, such as between the elongate body (201) and the sheath (203). In other embodiments, the expansion element (202) may be deployable into an expanded configuration via proximate control, e.g., using a pull wire, spring, inflatable balloon, or other suitable mechanism. In such cases, the sheath (203) may be optional. For example, the present disclosure includes embodiments wherein the expansion element (202) is configured to have a compressed configuration without the requirement or assistance of a constraining sheath (203). In some embodiments, the exterior surface of the elongate body (201) may include an inflatable portion, such as a balloon, between the elongate body (201) and the expansion element (202) for expanding the expansion element (202) into an expanded configuration. The expanded configuration may be uniform, e.g., having the same diameter along the entire length of the expansion element (202), but need not be. For example, the expansion element may expand such that each end opening is relatively wider or narrower than the interior portion, or one end opening may be relatively wider or narrower than the other end opening.

Any of the components of the device (200), e.g., the elongate body (201), expansion element (202), and/or sheath (203), may include one or more biocompatible materials. Non-limiting examples of suitable materials include silicone, rubber, metals, metal alloys, plastics, and polymers or polymer mixtures (e.g., polyethylene, polyurethane, polycarbonate, fluoropolymers, copolymers, etc.). Further, one or more components of the device (200) may include one or more coatings, e.g., to increase or decrease friction when moving with respect to another component of the device (200), another instrument, or a surface or passageway of the body.

Embodiments of the device (200) may include one or more flexible materials. For example, the sheath (203) may include a flexible material, such as a flexible polymer, to facilitate proximal or distal movement along the surface of the expansion element (202) and/or elongate body (201). In some embodiments, the flexibility of a distal portion of the sheath (203) and/or elongate body (201) may be greater than the flexibility of a proximal portion. For example, the flexibility of the device (200) may increase from a proximal end to a distal end to allow for greater range of movement at the distal end of the device (200).

The expansion element (202) may be in the form of a compressible mesh or cage, e.g., a mesh of one or more metals, metal alloys (e.g., nitinol), polymers, or a combination thereof. In some embodiments, the expansion element (202) may be formed of lasercut or braided metal. In other embodiments, the expansion element (202) may comprise a mesh of flexible polymer fiber.

Further, at least a portion of the expansion element (202) may include a biodegradable or bioresorbable material, such as a biodegradable polymer, to enable placement of the expansion element (202) in the body without the need for its removal. For example, at least a portion of the expansion element (202) may include a biodegradable material that dissolves in the body over a predetermined time, such as from several days or weeks to several months. In some embodiments, substantially all of the expansion element (202) may be biodegradable.

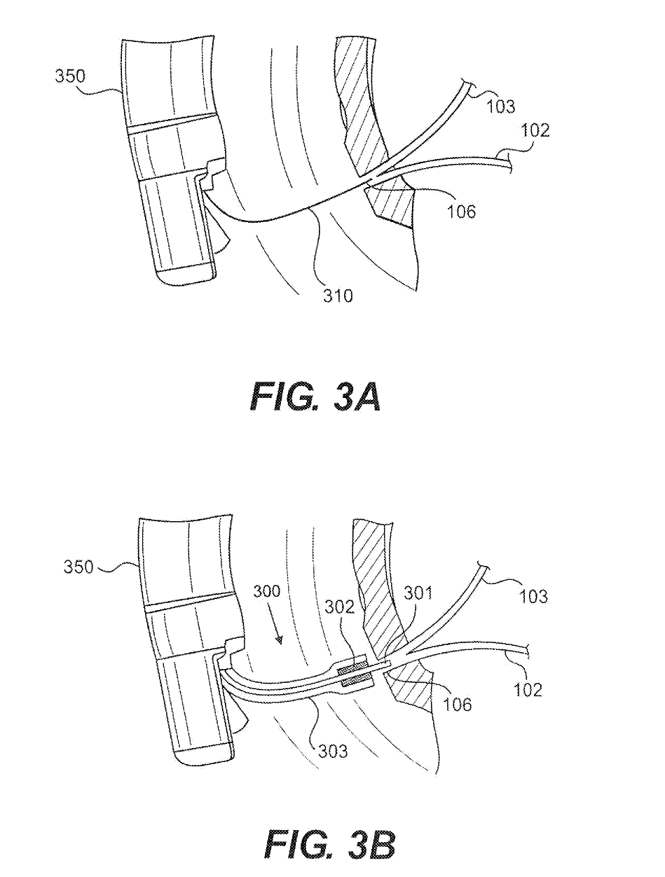

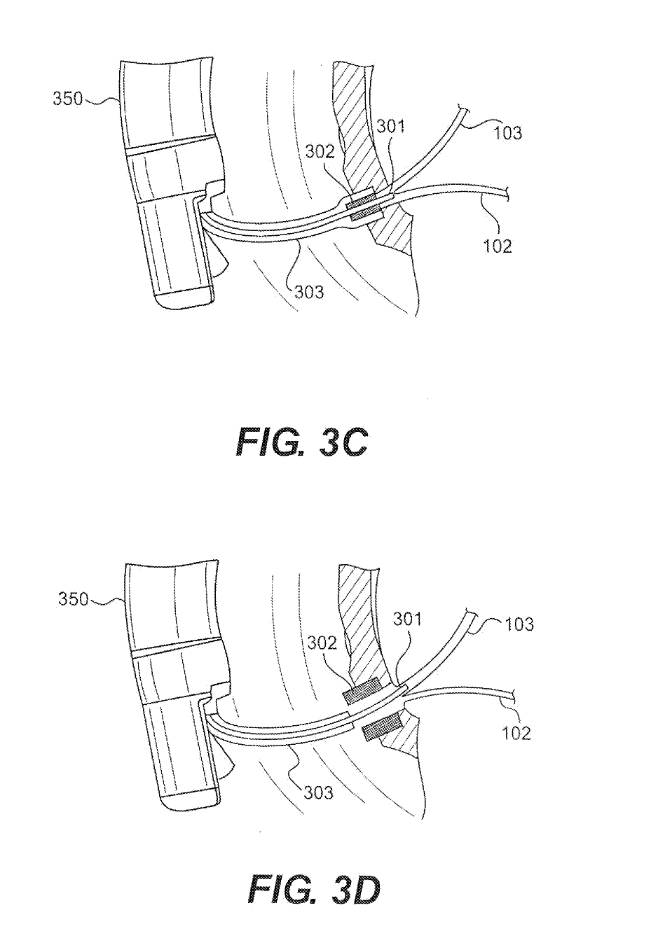

According to some embodiments of the present disclosure, an expansion device may be used in a medical procedure, such as an ERCP procedure. As illustrated in FIGS. 3A-3D, a device (300) may be introduced into the working channel of an endoscope (350) to reach the papilla (106) through an aperture of the endoscope (350). The endoscope (350) may include a proximal end and a distal end, and the working channel extending therebetween, wherein the aperture is located at the distal end of the endoscope (350). In at least some embodiments, the endoscope (350) may be a side-viewing endoscope, i.e., having a side aperture at the distal end, as shown in FIGS. 3A-3D. The side-viewing endoscope (350) may include a positioning mechanism such as, e.g., a ramp, elevator, or other feature to assist in deploying and/or orienting the expansion device (300) towards the papilla (106). The endoscope (350) may also include one or more proximal ports for receiving instruments such as the expansion device (300) in the working channel of the endoscope (350).

Referring to FIG. 3A, a guidewire (310) may pass through a working channel of the endoscope (350) to enter at least a portion of the papilla (106), e.g., to cannulate the papilla (106). Guidewires are available in a variety of diameters, e.g., ranging from about 0.018″ to about 0.035″ outer diameter, and typically include a solid metallic core with an applied coating. The coating may have markings for visual indicators, e.g., radiopaque markers, and may provide a lubricious surface for a catheter passed over the wire. The guidewire (310) may be of sufficient length to allow passage through the working channel of the endoscope (350), and the tip of the guidewire (310) may be tapered and/or constructed of a softer material to promote cannulation and minimize trauma to the patient.

Following cannulation with the guidewire (310), device (300) may be introduced into the endoscope (350) over the guidewire (310) as shown in FIG. 3B to enter at least a portion of the papilla (106). While FIGS. 3A-3B illustrate insertion of a guidewire (310) to cannulate the papilla (106), in some embodiments cannulation may be achieved without the use of a guidewire (310) by directly inserting the device (300) into the papilla (106). Device (300) shown in FIGS. 3B-3D includes an elongate body (301), an expansion element (302) in a compressed configuration located proximal to the distal most end of the elongate body, and a sheath (303) disposed along the length of the elongate body (301) and surrounding the expansion element (303) and the elongate body (301). FIG. 3B shows the distal most end or tip of the elongate body (301) entering the papilla (106) with the expansion element (302) and sheath (303) portion of the device (300) just outside of the papilla (106) within the duodenum (107).

As mentioned above, at least a portion of the device (300) may be flexible to facilitate orientation and/or insertion into the papilla (106). The distal end of the device (300), e.g., the distal end of elongate body (301) may include a tapered tip, typically ranging from about 3 Fr to about 6 Fr in diameter, to ease cannulation. In some embodiments, the device (300) may be steerable, e.g., to control movement of the distal end of the device (300). In some embodiments, the distal end of the device (300) may be deflected in one or more directions to align the distal most end or tip of the device (300) with the papilla (106).

In FIG. 3C, the device (300) has advanced further into the papilla (106) and along the ampulla (105) such that the compressed expansion element (302) and surrounding sheath (303) are disposed within the walls of the papilla (106). By moving the sheath (303) in a proximal direction, the sheath (303) uncovers the expansion element (302) allowing it to expand against the walls of the papilla (106) to widen or enlarge the opening as shown in FIG. 3D. The expansion element (302) may expand to a configuration having an opening sufficiently wide to allow for free passage of the elongate body (301), e.g., without contacting a tissue surface along the papilla (106) or ampulla (105). While FIGS. 3B-3D illustrate one mechanism for deploying the expansion element (302), e.g., self-expansion, other mechanisms may be used as discussed above.

When pressing against the papilla (106) walls, the expansion element (302) separates from the surface of the elongate body (301) to anchor the papilla (106) opening and allow the elongate body (301) to move freely along the ampulla (105) towards the pancreatic duct (102) and bile duct (103). The expansion element (302) may be configured for atraumatic contact with the walls of the papilla (106) to avoid damaging tissue, e.g., due to the materials used in the expansion element (302), its shape, and/or expansion mechanism. For example, the expansion element (302) may include a smooth outer covering.

Enlarging the papilla (106) with the expansion element (302) may allow for improved access and/or visualization of different features of the pancreaticobiliary system. For example, in some embodiments the elongate body (301) may include an optical device at the distal end to facilitate viewing of the pancreatic duct (102) and bile duct (103). Imaging may also be performed independently of the device (300), e.g., via an optical device introduced into a working channel of the endoscope (350) and advanced through the papilla (106). Expanding the papilla (106) may assist a physician in distinguishing the pancreatic duct (102) from the bile duct (103) and selectively enter the duct desired for examination, diagnosis, and/or treatment. For example, FIG. 3D shows the distal end of the elongate body (301) selectively entering the bile duct (103).

The elongate body (301) may include one or more lumens, e.g., for receiving guidewire (310) and injecting a contrast agent for fluoroscopy or other imaging analysis. For example, in some embodiments, the elongate body (301) is a sphincterotome. The sphincterotome may include an electrosurgical cutting wire at the distal end to enable deflection of the sphincterotome tip and to provide transmission of high frequency electrical current to incise the sphincter (108). In addition to aligning the sphincterotome with the papilla (106), deflection of the tip also may help to maintain contact with tissue of the ampulla (105) during incision, e.g., before placement of the expansion element (302). The physician may incise the sphincter (108) to gain access to the pancreaticobiliary system according to some embodiments of the present disclosure. In other embodiments, incision may not be necessary or desirable, e.g., if the expansion element (302) provides sufficient access without cutting tissue to enlarge the papilla (106).

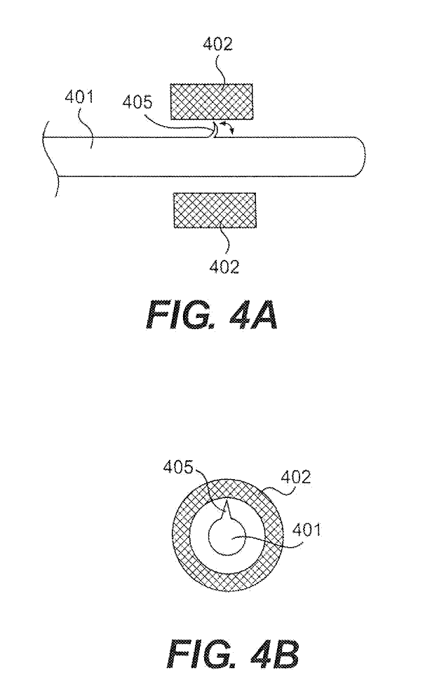

The expansion element may remain in place in the expanded configuration to anchor the papilla (106) as long as needed to complete a medical procedure. Removal of the expansion element may be achieved by any suitable mechanism. In an embodiment shown in FIG. 4A, for example, the elongate body (401) may include a protrusion such as a retractable hook (405) capable of extending radially outward from the surface of the elongate body (401). FIG. 4B shows a cross-sectional view of FIG. 4A, wherein the hook (405) extends radially outward from the elongate body (401). The retractable hook (405) may lie along the elongate body surface (401) until released or deployed, e.g., via a spring, pull wire, or other suitable control mechanism. Movement of the protrusion, e.g., hook (405), may be reversible, e.g., the hook (405) may be selectively extended and retracted. When extended towards the expansion element (402), the hook (405) may catch a portion of the expansion element (402) as the elongate body (401) moves proximally through the papilla (106) towards the duodenum (107), thus removing the expansion element (402) from the papilla (106). In other embodiments, the expansion element (402) may include a protrusion to be received by the elongate body (401) to facilitate removal of the expansion element (402) from the papilla (106).

One or more other removable mechanisms may be used. For example, the expansion element (402) may be removed by extending a grasper through an endoscope working channel and grasping a proximal portion of the expansion element (402) for removal. In some embodiments, the expansion element (402) may be compressed prior to removal. Further, as discussed above, the expansion element (402) may include a biodegradable material such that removal of the expansion element (402) may not be necessary.

Any of the features discussed herein in connection to an embodiment may be used in combination with one or more features of any other embodiment. Further, other embodiments of the present disclosure will be apparent to those skilled in the art from consideration of the specification and practice of the embodiments disclosed herein. It is intended that the specification and examples be considered as exemplary only, with a true scope and spirit of the present disclosure being indicated by the following claims.

We claim:

1. A method of accessing the pancreaticobiliary system, the method comprising:
introducing a distal end of an instrument into at least a portion of a duodenal papilla, the instrument including:
an elongate body having a proximal end, a distal end, and at least one lumen extending therebetween; and
an expansion element circumferentially disposed around the elongate body, the expansion element having a compressed configuration and an expanded configuration; and
deploying the expansion element into the expanded configuration, wherein in the expanded configuration, the expansion element expands against a wall of the papilla.

2. The method of claim 1, further comprising introducing a guidewire into at least a portion of the papilla before introducing the instrument into the papilla.

3. The method of claim 1, wherein the expansion element is self-expanding.

4. The method of claim 3, wherein the instrument further includes a sheath circumferentially disposed around the expansion element, and wherein deploying the expansion element includes moving the sheath in a proximal direction to uncover the expansion element.

5. The method of claim 1, wherein the expansion element includes a metal mesh.

6. The method of claim 1, wherein the elongate body is a sphincterotome having an electrosurgical cutting wire.

7. The method of claim 1, further comprising advancing the elongate body in a distal direction.

8. The method of claim 1, further comprising using an optical device to image a feature of the pancreaticobiliary system.

9. The method of claim 1, further comprising performing a medical procedure within at least one of the pancreatic duct and the bile duct with the treatment instrument.

10. The method of claim 1, further comprising removing the expansion element from the papilla.

11. A medical device comprising:
an elongate body having a proximal end, a distal end, and at least one lumen extending therebetween, the elongate body configured for insertion into a duodenal papilla; and
an expansion element circumferentially disposed around the elongate body, the expansion element having a compressed configuration and an expanded configuration suitable for insertion into and enlarging the duodenal papilla, wherein a distal end of the expansion element is located proximal to the distal end of the elongate body.

12. The medical device of claim 11, further comprising a sheath circumferentially disposed around the expansion element and the elongate body, wherein the sheath is slidable with respect to the expansion element.

13. The medical device of claim 11, wherein the expansion element includes a metal, a polymer, or a combination thereof.

14. The medical device of claim 11, wherein the expansion element includes an expandable metal mesh.

15. The medical device of claim 11, wherein the expansion element includes a coating.

16. The medical device of claim 11, wherein the expansion element is fixed to the elongate body.

17. The medical device of claim 11, wherein the expansion element is movable with respect to the distal end of the elongate body.

18. The medical device of claim 11, wherein the elongate body includes a retractable hook for grasping a portion of the expansion element.

19. The medical device of claim 11, wherein the expansion element is configured for atraumatic contact with a tissue surface in the expanded configuration.

20. The medical device of claim 11, wherein the elongate body is a sphincterotome having an electrosurgical cutting wire.
